(19) United States
(12) Patent Application Publication
(10) Pub. No.: US 2011/0294208 A1
(43) Pub. Date: Dec. 1, 2011

(54) METHOD AND DEVICE FOR CELL SELECTION AND COLLECTION IN AN ISOLATED CULTURING ENVIRONMENT

(76) Inventors: Nancy Allbritton, Chapel Hill, NC (US); Christopher Sims, Chapel Hill, NC (US); Wei Xu, Chapel Hill, NC (US)

(21) Appl. No.: 13/145,917

(22) PCT Filed: Feb. 11, 2010
(86) PCT No.: PCT/US2010/023854

§ 371 Date: Aug. 9, 2011

Related U.S. Application Data
(60) Provisional application No. 61/151,652, filed on Feb. 11, 2009.

(51) Int. Cl. C12N 5/16; C12M 1/00; C12N 5/071; C12M 1/12; C12N 1/20; C12N 1/14; C12N 1/16; C12M 3/00; C12N 5/075

(52) U.S. Cl. 435/346; 435/289.1; 435/395; 435/252.1; 435/254.1; 435/255.1; 435/297.1

(57) ABSTRACT

An apparatus for collecting or culturing cells or cell colonies has a common substrate and a plurality of cell carriers releasably connected to the common substrate. The carriers are arranged in the form of an array. The invention employs microcups as the cell carriers. The substrate is preferably free of barriers between the microcups, and in some embodiments the microcups have porous walls. Methods of using the apparatus are also described.

This invention was made with Government support under grant number EB007612 from the National Institutes of Health. The US Government has certain rights to this invention.

### BACKGROUND OF THE INVENTION

The collection of individual cells and colonies using a releasable microfabricated elements array has been disclosed in U.S. patent application Ser. No. 60/744,579 “Efficient Collection of Single Cells and Colonies of Cells from Arrays” (see also WO2007/118208). The fabrication of a plate of micropallets has been disclosed in U.S. patent application Ser. Nos. 11/243,926 “Micropatterned Plate With Micro-Pallets for Addressable Biochemical Analysis” (see also US2006-0121500), and 60/746,008 “Method of Manufacture of a Plate of Releasable Elements and its Assembly into a Cassette” (see also WO2007/127990). In brief, cells can be grown on an array of microfabricated releasable micropallets. Individual micropallets and the cell or colony attached to the micropallet can be released and collected for further culture or analysis. To create a micropallet array, a plate of micropallets can be manufactured by optical lithography and photosensitive polymer, or by optical lithography and etching, or by the use of a stencil, or by the use of a laser, or by the use of machining material, or by the use of molding a polymer.

A plate of micropallets surrounded with gas walls has been disclosed in U.S. patent application Ser. No. 11/539,695 “A Micro-Bubble Plate for Patterning Biological and Non-biological Materials” (see also US 2007-0128716 A1). The gas bubbles are trapped in the cavities between the micropallets, thus forming a continuous gas wall surrounding the micropallets. The gas bubbles act to localize the attachment of biological materials (such as biomolecules or cells) and non-biological materials only on the top surface of micropallets.

The provision of barriers, be they formed of gas bubbles or other materials, requires additional process steps in the construction of the apparatus, and/or modification or derivitization of substrate surfaces that decreases the stability and shelf-life thereof. However, there has not been a way to separate cells from one another on individual carrier elements (or micropallets) within the array without forming such barriers on the substrates between the micropallets.

### SUMMARY OF THE INVENTION

A first aspect of the present invention is, in an apparatus for collecting or culturing cells or cell colonies, the apparatus comprising a common substrate and a plurality of cell carriers releasably connected to the common substrate, with the carriers arranged in the form of an array, the improvement comprising: employing microcups as the cell carriers, each of the microcups comprising a base portion and a top portion, the top portion having a cavity configured to contain cells formed therein.

A second aspect of the invention is, in a method of collecting or culturing cells or cell colonies by (a) depositing a liquid media carrying the cells on an apparatus comprising a common substrate and a plurality of cell carriers releasably connected to the common substrate, with the carriers arranged in the form of an array, and then (b) permitting or allowing the cells to settle on or adhere to the cell carriers, the improvement comprising: employing microcups as the cell carriers, each of the microcups comprising a base portion and a top portion, the top portion having a cavity configured to contain cells formed therein.

In some embodiments of the invention, the array is a barrier-free array (i.e., the array is characterized by open gaps between the microcups, without substrate walls or substrate barriers formed on or connected to the substrate and positioned between the microcups).

In some embodiments of the invention, wherein the cells are deposited on the apparatus at an efficiency of capture of at least 50, 60, 70, 80 or 90 percent.

In some embodiments of the invention, the cells are non-adherent cells, such as hybridomas, lymphocytes, stem cells, egg cells or oocytes, gram negative bacteria, and gram positive bacteria, yeast and fungi.

In some embodiments of the invention, the microcups are transparent.

In some embodiments of the invention, the microcups in the array are separated by gaps, and wherein the gaps have an average width of from 2 to 200 micrometers.

In some embodiments of the invention, wherein the gaps have an average width of not more than 5, 10, 100, 500, or 1000 micrometers.

In some embodiments of the invention, wherein the array is in the form of an interdigitated array.

In some embodiments of the invention, wherein the apparatus is essentially free of walls or barriers formed on the common substrate between the cell carriers.

In some embodiments of the invention, the substrate is uncoated and underivatized.

In some embodiments of the invention, the microcups have heights in the range of 2 micrometers up to 400 or 500 micrometers.

In some embodiments of the invention, the microcups have maximum widths in the range of 5 micrometers up to 1000 micrometers.

In some embodiments of the invention, the cavity has a depth of from 1 to 200 micrometers.

In some embodiments of the invention, the cavity has a minimum width of at least 1 micrometer.

In some embodiments of the invention, the cavity has a maximum width of less than 1000 micrometers.

In some embodiments of the invention, the cavity is cylindrical, elliptical, triangular, rectangular, pentagonal, or hexagonal in shape.

In some embodiments of the invention, the cavity comprises side wall portions, and wherein the side wall portions are parallel, inwardly tapered, or outwardly tapered.

In some embodiments of the invention, the cavity comprises side wall portions, and the side wall portions have at least one cell-retaining member formed thereon.

In some embodiments of the invention, the microcups comprise a photoresist resin.

In some embodiments of the invention, the microcup top portion comprises a cavity wall defining, the cavity, and wherein the cavity wall completely surrounds the cavity.

In some embodiments of the invention, the microcup top portion comprises a cavity wall defining the cavity, and wherein the cavity wall partially surrounds the cavity.

### BRIEF DESCRIPTION OF THE DRAWINGS

FIG. 1. Schematic of microcup array design. Views (a), (c), (e), (g), (i), (k) and (m) are top views, and views (b), (d), (f), (h), (j), (l) and (n) are side views.

FIG. 2. Images of microcup arrays: (a) top view of microcup array (b) photo of microcup array with a region of the microcups removed (c) photo of a single microcup released from the array

FIG. 3. Microscopic pictures of Ba/F3 cells (a) and HeLa cells (b) loaded into microcups on the array. (c) Image by bright-field microscopy of three HeLa cells attached to the bottom of a microcup. (d) A released microcup with HeLa cells retained inside. (e) A released microcup and a collected microcup (f) with a Ba/F3 cell retained inside.

FIG. 4. Cell capture efficiency of microcups

FIG. 5. (a) Microscopic picture of microcups with the cell culturing medium between microcups removed while the cell medium inside the microcup is retained. BAF3 cells were loaded into microcups before the inter-cup medium was removed. (b) Detailed microscopic picture of microcups with inter-cup medium removed.

FIG. 6. Fabrication of microcup arrays. (A) Schematic of fabrication process flow for the microcup array. (B) Transmitted light photomicrograph of a section of a completed microcup array. (C) ESEM photo of the same array.

FIG. 7. Cell plating on the microcup arrays. (A) Photomicrograph of Ba/F3 cells plated on a micropallet array lacking a virtual wall. Under these conditions all cells settled into the regions between the pallet structures and none were seen on the upper surfaces of the pallets. (B) Ba/F3 cells plated on a microcup array. Cells were present both within the microcup and in the inter-cup regions.

FIG. 8. Characterization of factors influencing microcup cell capture efficiency. (A) Plot of capture efficiency (percentage of total cells captured inside the microcup) vs. the area of the array covered by microcups. (B) Plot of capture efficiency vs. ratio of number of cells plated/number of microcups. (C) Plot of capture efficiency vs. microcup edge thickness.

FIG. 9. Improved microcup design. (A) Photomicrograph of a Ba/F3 cell whose diameter is ˜20 μm trapped inside the central region of an array composed of microcups with 70 μm sides and 15 μm gap. (B) Photomicrograph of Ba/F3 cells plated at high density relative to the number of microcups on the array (n_{cell}=40,000, n_{microcup}=5,000) demonstrating the trapping of cells at the border region of four neighboring microcups. The focal plane corresponds to the base of the microcup structure so that cells within the microcup are out of focus. (C) Schematic comparing the maximal gap distances between the square and an interdigitated microcup design. (D) Photomicrograph of Ba/F3 cells captured in the interdigitated microcups. No cells were observed to be present at the border regions of three neighboring microcups. (E) Plot of density of cells plated vs. capture efficiency using the interdigitated microcup design.

FIG. 10. Cell growth on microcup arrays. (A) Transmitted light and fluorescence photomicrographs of wild type Hela cells cultured on a microcup array for 72 hrs and then labeled with Calcein Red-Orange. (B) Transmitted light and fluorescence photomicrographs of single HeLa cells captured in adjacent microcups 1 hr after plating (microcup array dimensions identical to “A”). The cell in the center microcup possesses a fluorescent nucleus due to stable expression of a GFP/histone-H1 fusion protein. C) Transmitted light and fluorescence images of the same region of the microcup as in “B” after cells have been in culture for 96 hrs.

FIG. 11. Collection and clonal expansion of single cells from a microcup array. A & B) Photomicrographs of an individual HeLa cell before and after release of its microcup from an array. (C) Photomicrograph of a microcup containing a single HeLa cell after release and collection. (D) Photomicrograph of a clonal colony generated by collection of a single HeLa cell from a microcup array.

FIG. 12 A-E schematically illustrate representative examples of porous wall designs for carriers of the present invention. As will be seen, the continuous wall, the barrier can contain openings of any shape or size, can contain perforations of any shape (A-B), can be formed by posts (C-D), or the wall can be manufactured of a porous material or a hydrogel (E).

### DETAILED DESCRIPTION OF THE INVENTION

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

Like numbers refer to like elements throughout. In the figures, the thickness of certain lines, layers, components, elements or features may be exaggerated for clarity. Where used, broken lines illustrate optional features or operations unless specified otherwise.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening, elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

Spatially relative terms, such as “under,” “below,” “lower,” “over,” “upper” and the like, may be used herein for ease of description to describe an element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus the exemplary term “under” can encompass both an orientation of over and under. The device may otherwise be oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only, unless specifically indicated otherwise.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section. Thus, a first element, component, region, layer or section discussed herein could be termed a second element, component, region, layer or section without departing from the teachings of the present invention. The sequence of operations (or steps) is not limited to the order presented in the claims or figures unless specifically indicated otherwise.

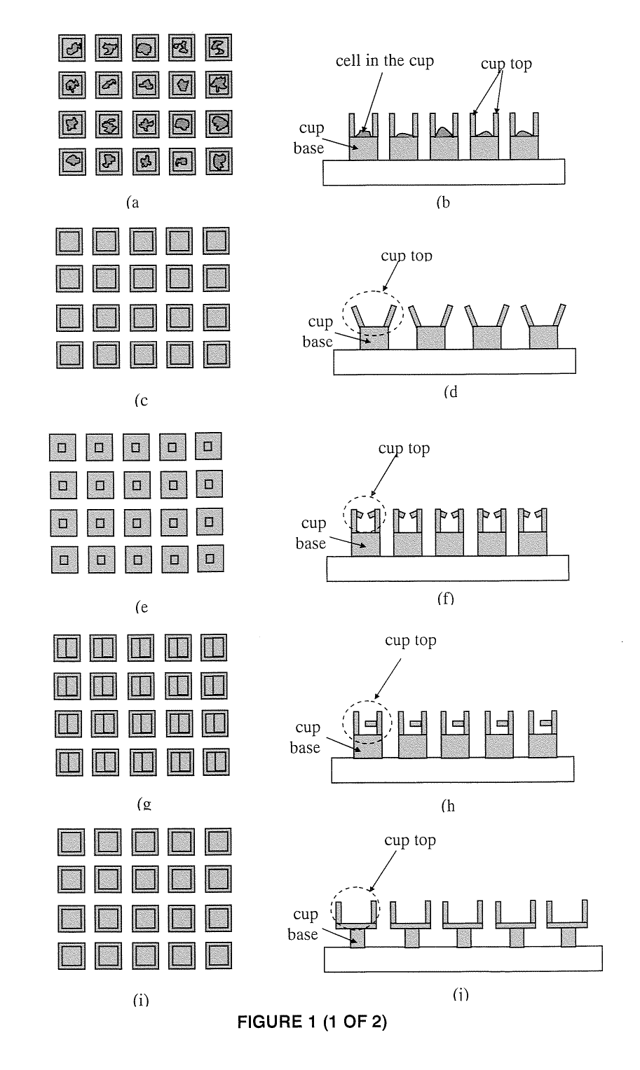

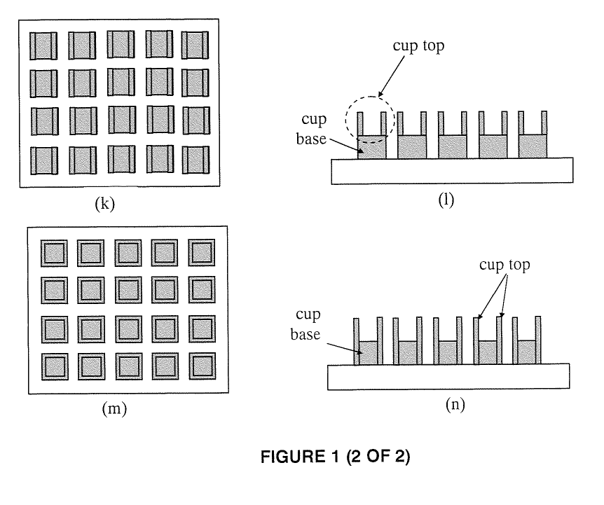

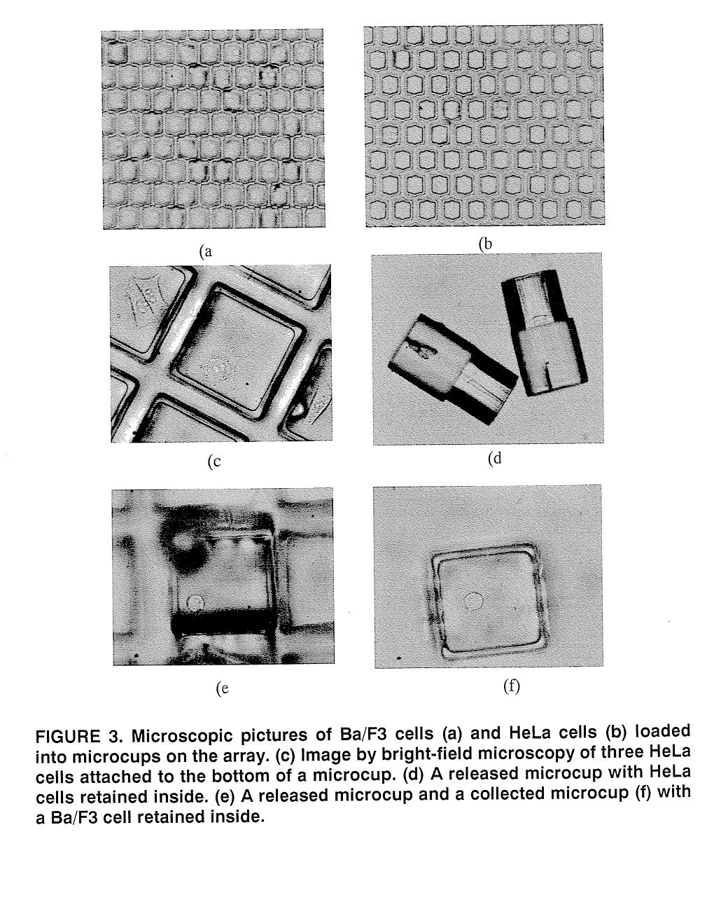

“Interdigitated” as used herein with respect to carriers or microcups in an array means that the pattern of the array is staggered or off-set (typically in a uniform or repeating, pattern) so that gap intersections are reduced in size and the opportunity for cells to settle at such intersections is reduced. For example, the arrays in FIG. 1 are not interdigitated (note the presence of repeating 4-armed intersections, or intersecting parallel gaps), while the arrays in FIG. 3 are interdigitated (note the presence of three-armed intersection of gaps, and the presence of a pair of oppositely facing, vertical interdigitating ridges on the outer side wall of each microcup that aligns with a horizontal gap between off-set microcups in each adjacent row.). Interdigitation can be achieved by one or more of a variety of means. The microcups can be hexagonal or triangular in cross-section; the microcups, when square or rectangular, can be offset from one another in adjacent rows (see, e.g., FIG. 3). The microcups can be provided with one or more vertical ridges that, when arranged in an array, interdigitates with gaps between microcups in adjacent rows. Numerous variations on the foregoing, will be apparent to those skilled in the art.

“Cells” for carrying out the present invention are, in general, live cells, and can be any type of cell, including animal (e.g., mammal, bird, reptile, amphibian), plant, or other microbial cell (e.g., yeast, gram negative bacteria, gram positive bacteria, fungi, mold, algae, etc.).

“Liquid media” for carrying out the present invention, in which cells are carried for depositing on an array as described herein (and specifically within the cavities of the microcups) may be any suitable, typically aqueous, liquid, including saline solution, buffer solutions, Ringer's solution, growth media, and biological samples such as blood, urine, saliva, etc. (which biological samples may optionally be partially purified, and/or have other diluents, media or reagents added thereto).

“Substrate” as used herein may comprise, consist of or consist essentially of any suitable material, which is preferably optically transparent or optically transmissive. Examples of suitable materials include, but are not limited to, glass such as pyrex glass, quartz, silica, aluminum, sapphire, silicon, PMMA, and combinations thereof. See, e.g., U.S. Pat. No. 7,489,837.

“Photoresist” as described herein, in connection with microcups which comprise, consist of or consist essentially of a photoresist resin or polymer, may be any suitable material, which is preferably optically transparent or optically transmissive. Examples of suitable materials include, but are not limited to, novolac-based photoresists, polyvinylphenol-based photoresists, t-BOC derivatives of polyvinylphenol-based photoresists, etc. See, e.g., U.S. Pat. Nos. 7,385,751 and 6,788,452.

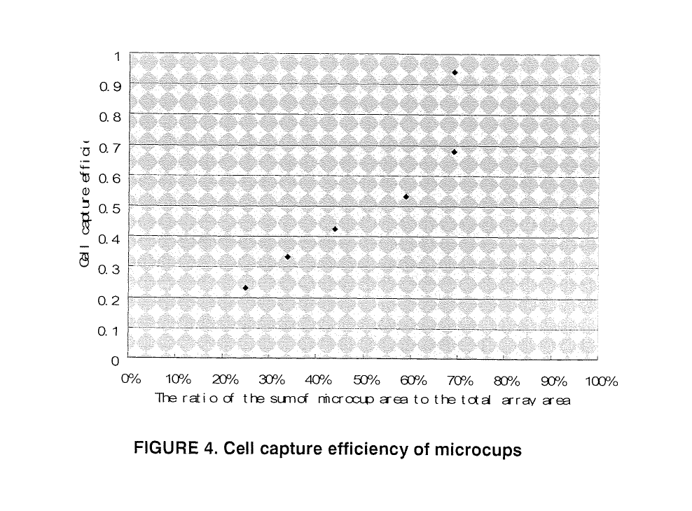

The present invention describes a method and apparatus for culturing and subsequently collecting cells using an array of microfabricated elements. These elements, known herein as microcups enable single or small numbers of cells to be cultured in a miniaturized culture well that isolates the cell(s) cultured in the microcup from other cells cultured on the array. Furthermore, these microcup elements are releasable from the underlying substrate to enable collection of materials, including living cells, from the array. An important part of this invention over a previous technology (the micropallet array—see above) is that the releasable microcups can be used to create “wall-less” or “barrier-free” arrays or arrays in which no physical barrier is needed between the releasable elements in order to direct materials or cells onto the upper face of the individual elements. In other words, the design obviates the need for a microbubble (aka “virtual wall”) or other means for geographically restricting growth of cells to the individual elements, such as a continuous wall (or surface modification) formed by a material that resists cell adhesion poly(ethylene glycol)). The efficiency of capturing cells in the microcups is linearly related to the ratio of the sum of the surface area of the microcup culture region to the total area of the array when the gap of microcup is larger than the cell. However, when the gap is reduced in size similar to the cell diameter, cell capture efficiency is highly increased and no longer follows this linear relationship (FIG. 4).

Similar to other devices designed to capture or culture cells in microwells, the microcups provide a geographically restricted cell culturing environment; however, novel to this invention is the ability to release and collect individual microcups and cells retained within the microcup so that further analysis or growth separated from the array is possible. In addition, the micron-scale walls of the microcups provide an environment that can be partially or completely isolated from the fluidic microenvironment of neighboring cells. This capability provides the potential of creating cell niches in which cells can be subsequently collected and analyzed or expanded. Also, the current invention provides a means for selection and collection of both adherent and nonadherent cells as the sides of the microcup act to retain cells in the cup-like structure without the requirement for the cells within the microcup to be strongly adhered within the microcup during the release and collection process. In this description, culture and collection of nonadherent and adherent cells are shown as a non-limiting illustrative embodiment of the invention.

This invention is made possible by the use of a microfabricated plate composed of the releasable microfabricated microcup elements. The design of the microcup array is further specified as a patterned plate composed of, but not limited to, two layer microstructures including base and cup edge. A single layer may also be used to form the cups. In the illustrative embodiment, the array lacked intervening structures between the microcups or groups of microcups, such as regions of air or solid structures such as walls composed of poly(ethylene glycol) or a photoresist (e.g. SU-8 or 1002F). Nevertheless, such structures are envisioned and could be of the same or different materials from the material(s) forming the microcups. If, as in the illustrative embodiment, there are no walls created between microcups, liquid such as cell culture medium fills the gap between the microcups. Once the cells are loaded into the microcups, the cells are geographically restricted to the inside of the cup which remains open at its upper aspect. The sides of the microcups act as barriers to cell migration from microcup to microcup. While geographically restricted, cells on the array share a similar fluidic environment when the fluid level covering the array is above the height of the microcup sides. Nevertheless, under this circumstance, the sides of the cup do provide a partial barrier for the diffusion of substances from one microcup to another via the fluid environment. If the purpose of the microcup application is to provide a totally isolated culture environment for cells, two means are envisioned to accomplish this degree of isolation. The fluid level outside the microcups can be lowered below that of the sides of microcups. Under this condition, fluid inside the microcups will be retained within the microcup due to the cup-like nature of the design. Also, a cover can be placed in close apposition to the upper edges of the microcups on the array to prevent fluid exchange or diffusion of soluble substances among the microcups.

The present invention can be practiced without the need for an intervening barrier of gas or other material to localize cells to the culture surface of the microfabricated elements.

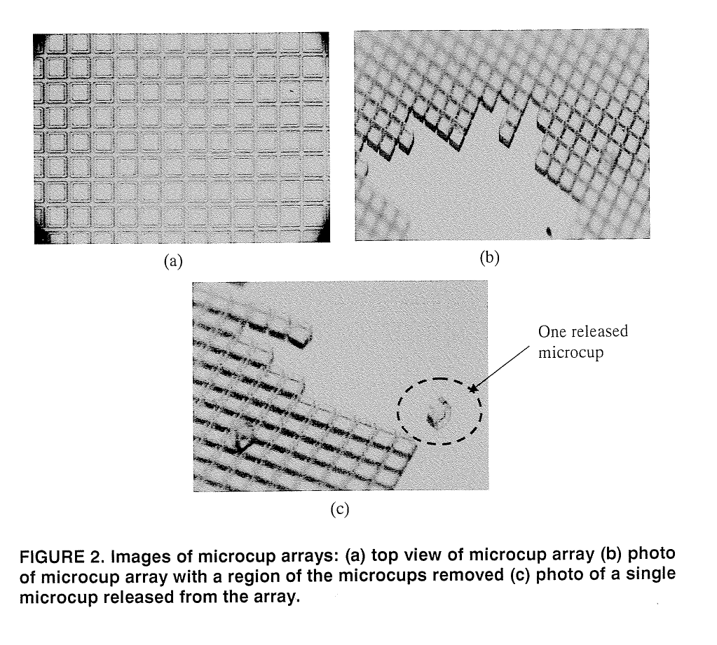

An overview of examples in the design of a microcup array plate is shown in FIG. 1. An array composed of microcups is created on a plate. The size, the shape, the surface roughness, the surface pattern and the layers of the microcups can be similar or can vary. Within each individual microcup on the plate, the dimension, shape and the material of the different layers can be similar or can vary. As stated above, while not necessary microfabricated structures such as walls could be present between the microcups. The height of the structures between the microcups can be made the same, lower or higher than the height of the microcups. These structures could be made by a variety of materials such as, but not limited to, a gas (e.g. air), a hydrogel (e.g. poly[ethylene glycol]), a polymer (e.g. the photoresists 1002F or SU-8), or a combination of these materials. When no structure is present between the elements of the array, fluid such as cell culture medium will fill the gap between the microcups. The spacing between the microcups can be varied to meet the unique requirements of different applications. Photomicrographs of microcups created from SU-8 photoresist are shown in FIG. 2.

An illustrative embodiment of the invention was demonstrated by the application of a microcup array to selecting and collecting adherent cell types (HeLa) and nonadherent cell types (Ba/F3). While Ba/F3 and HeLa were used as a model cell types, other cell types including human stem cells and primary cells adult and embryonic stem cells, cells taken directly from patients, or from animal models) tissue culture cell lines, plant cells, yeast cells, and other cell types could be used. Various cell types can be obtained from human or animal sources such as, but not limited to embryo or tissue sources, or they can be originated from plant tissues.

Cells are loaded and cultured on the microcup array by adding a suspension of cells and allowing the cells to settle or attach to the bottom of microcups. By controlling the density of the cell suspension, single or multiple cells can be positioned inside the microcups. It is also possible to deposit cells directly into the microcups by pipet, fluid flow, dielectrophoresis, magnetic manipulation, or other cell manipulation techniques.

The arrays in this non-limiting example were designed so that there were no walls or other structures between the microcups. For these studies, the gap between microcups was designed to be comparable to the size of the cells in order to increase the number of cells dropping into the microcups instead of settling into the gap between the microcups. The gap size could also be of greater magnitude if desired depending on different applications. In experiments studying the percentage of cells captured inside the microcup elements as gap size was varied, it is found the capture efficiency was linearly related to the ratio of the total surface area of the microcup culture area to the total area of the array (FIG. 4). As expected when the total surface area of the microcup culture region was near that of the total array area, most cells were captured within the microcups. When the gap between microcups is similar to the cell size, the cell capture efficiency does not follow a linear relationship as it was seen to dramatically increase at a defined point as the ratio of microcup area to total array area increased. In this condition, the shape of the microcup also impacted capture efficiency. The original microcup design was of a simple four-sided square (FIG. 2). A more sophisticated interdigitated design (FIG. 3) was shown to improve capture efficiency due to the more consistent gap width throughout the array. Using this design, a capture efficiency greater than 90%, (FIG. 4), was obtained versus 69% for an array of square cups of similar microcup to total surface area ratio.

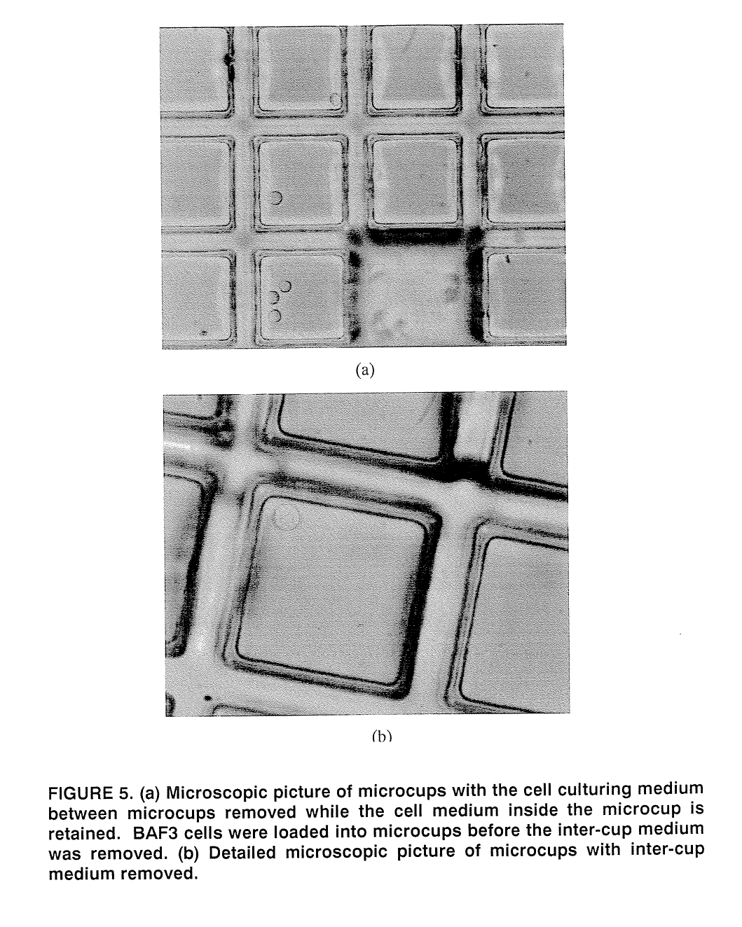

Once the cells settle into the microcups, the cell culture medium outside the microcups could be removed while leaving the cells in the microcups (FIG. 5). This provides one means to isolate the cells within individual microenvironments on the array. After capture and/or culture of cells on the array, cells of interest can be identified by a variety of techniques for cell analysis, most commonly microscopic imaging techniques, including but not limited to bright-field, phase contrast, Kohler illumination, fluorescence, and others. Microcups in which cells of interest were found were released using a pulsed Nd:YaG laser as previously described by the investigators (FIG. 3). Cells collected in this manner could be used for a variety of purposes including culture for generating a clonal cell line or analysis by any means (e.g., PCR, Southern blot, or other analysis technique). The analyses may cover a broad range of genomics, proteomics, biochemical or other study and can be performed using any of a variety of existing or future technologies.

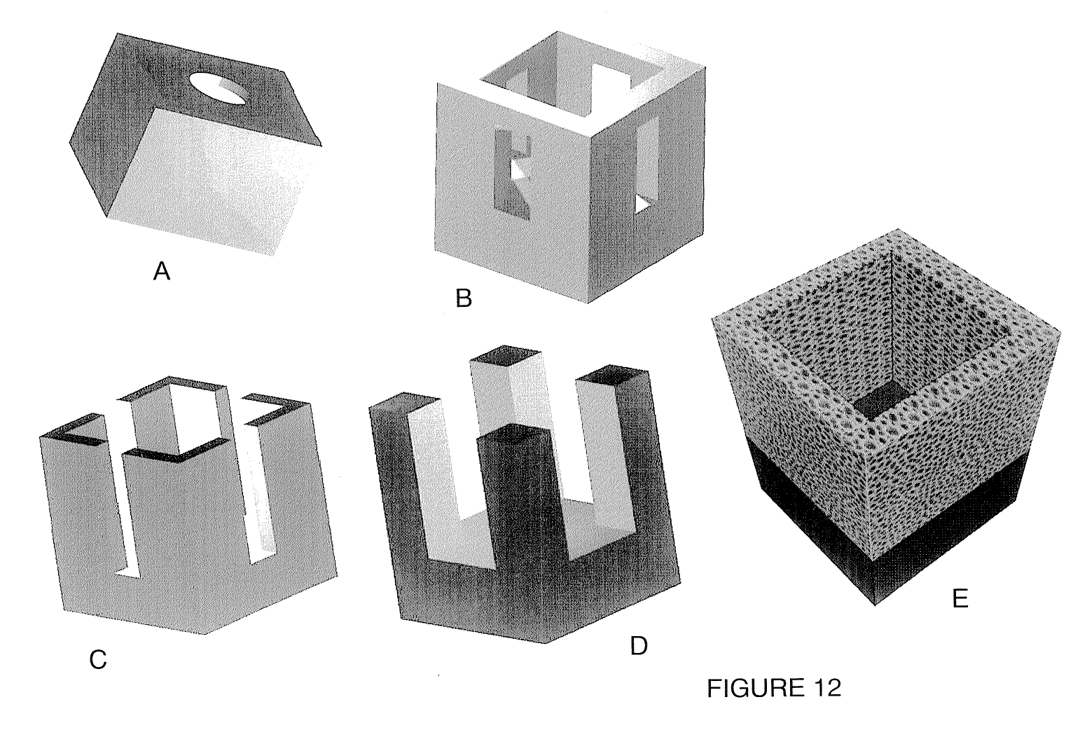

Porous cavity walls. In some embodiments of the invention, the cavity is defined by side wall portions that are porous or permeable, as shown in FIG. 12 A-E. The walls may be porous due to the formation of openings such as vias, windows, openings, fenestrations, etc., therein, which openings may be oriented vertically, horizontally, or in any other suitable manner. In other embodiments, the wall itself may be formed of a permeable or semipermeable material such as a hydrogel (which may or may not further include physical openings formed thereon). In general, the wall openings, and/or the permeable material from which the walls are formed, are configured so that cells (particularly live animal cells) are retained in the cavity, but nutrients in the tissue culture medium in which the microcup is immersed pass through the wall so that survival or viability of cells in lower portions of the cavity (e.g., when the cavity a depth of at least 20, 50, 80 or 100 micrometers, up to 200, 500 or 1000 micrometers or more) is enhanced. Where such porous cavity walls are employed, it is preferred that the array is a barrier-free array (that is, or for example, one in which the apparatus is essentially free of walls or barriers formed on the common substrate between the cell carriers), and which preferably provides a configuration in which gaps are included between the carriers as described above so that nutrients can pass from a media in the gaps outside the carriers through the porous walls to the cells contained in the cavities within the carriers.

### Experimental

### Microcup Arrays for the Efficient Isolation and Cloning of Adherent Cells

Described herein is a technique that combines the capabilities and advantages of the microwell and micropallet array formats. A two-step process is used to produce arrays of releasable micron-scale cups on a glass substrate. Both adherent and non-adherent cells are utilized to demonstrate the cell capture capability of the microcup array. The efficiency of capturing cells as a function of percent total surface area formed by the cups is determined and designs to enhance capture efficiency are studied. Culture of viable cells within the cups is demonstrated by viability assay and by generating and maintaining clonal colonies in culture. Isolation of individual viable cells is demonstrated by highly efficient single-cell cloning.

Materials. SU-8-50 photoresist and SU-8 developer (1-methoxy-2-propyl acetate) were purchased from MicroChem. Corp. (Newton, Mass., USA). UVI-6976 photoinitiator (triarylsulfonium hexafluoroantimonate salts in propylene carbonate) was purchased from Dow Chemical (Torrance, Calif.) and poly(dimethylsiloxane) (PDMS) (Sylgard 184 silicone elastomer kit) was purchased from Dow Corning (Midland, Mich.). EPON resin 1002F (phenol, 4,4′-(1-methylethylidene)bis-, polymer with 2,2′-[(1-methylethylidene) bis(4,1-phenyleneoxymethylene]bis[oxirane]) was obtained from Miller-Stephenson (Sylmar, Calif.). All other photoinitiators and resins were from Sigma-Aldrich (St. Louis, Mo.) as were γ-butyrolactone (GBL). (Heptadecafluoro-1,1,2,2-tetrahydrodecyl) trichlorosilane was from Gelest Inc. (Morrisville, Pa.). To prepare masks for microcup fabrication, the patterns were first drawn using TurboCAD (IMSI/Design, LLC, Novato, Calif.) and then sent to Fineline Imaging (Colorado Springs, Colo.) for printing and fabricating the final chrome mask. Dulbecco's Modified Eagle Medium (DMEM), RPMI 1640 medium, fetal bovine serum (FBS), 0.05% Trypsin-EDTA 1X and penicillin/streptomycin were obtained from Invitrogen (Carlsbad, Calif.). Fibronectin, glass microslides and all other reagents were obtained from Fisher Scientific (Pittsburgh, Pa.).

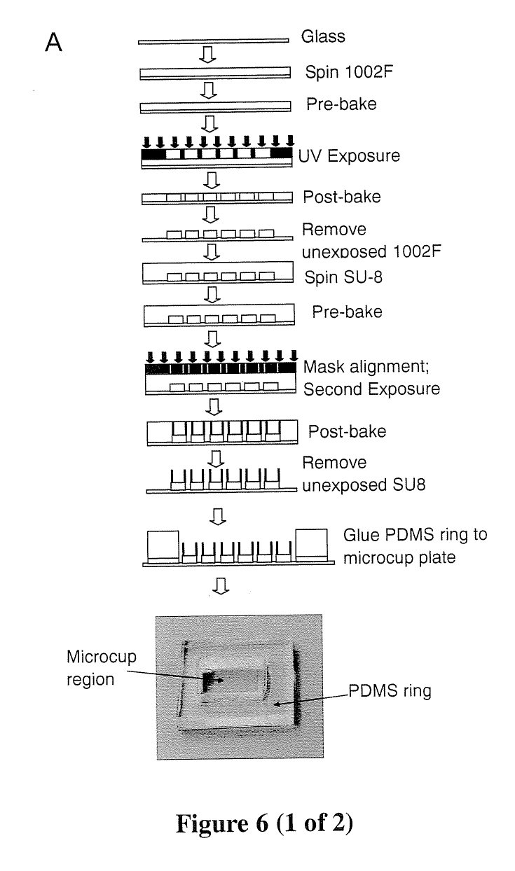

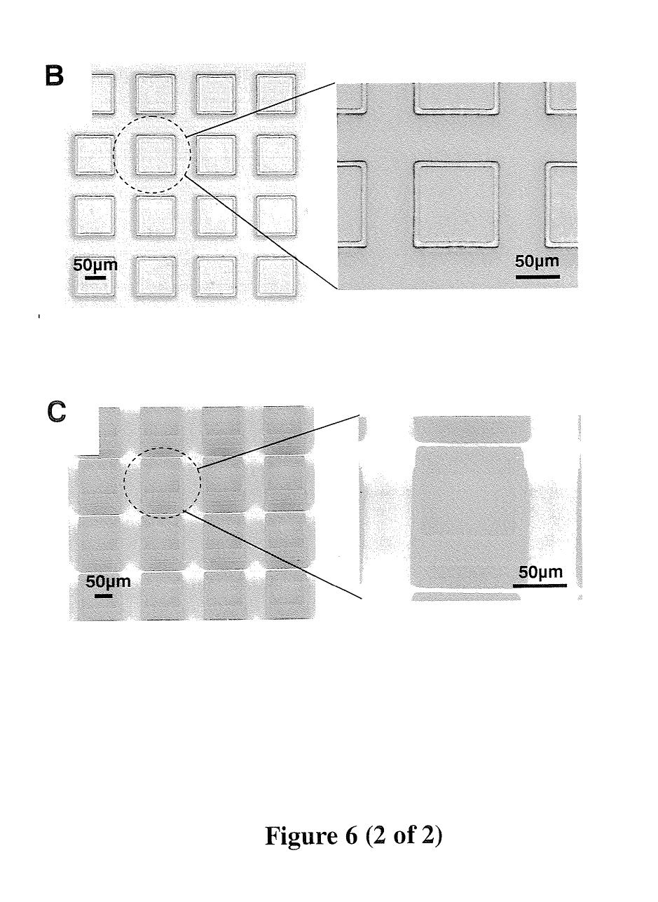

Fabrication of the micro-cup arrays. The fabrication of the microcup used a standard multilayer microfabrication process as shown in FIG. 6. First 1002F-50 photoresist was prepared as previously described, and was then spun on glass slides using a spin coater (WS-400B-6NPP/LITE, Laurell Technologies Corporation, North Wales, Pa.) to create a 50 μm thick photoresist film by two step spinning: an initial spin of 10 s at 500 rpm spin speed followed by a second spin of 30 s at 2200 rpm (J.-H. Pai et al., Anal. Chem., 2007, 79, 8774-8780). The coated slides were then soft baked in a gravity flow convection oven (Isotemp Oven, Fisher Scientific, Pittsburgh, Pa.) at 95° C. for 45 mins to remove organic solvent. After baking, the slides were allowed to cool to room temperature (˜40 mins). The first layer of the microcup structure, i.e. the cup base, was prepared by exposing the coated slides under cup base chrome mask using a collimated UV source (76 mW/cm², Oriel, Newport Stratford, Inc., Stratford, Conn.). The post exposure bake (PEB) was performed by baking exposed slides in the convection oven for 10 mins. The slides were then cooled to room temperature. After cooling, the slides were developed in a bath of SU-8 developer in a 100 mm glass Petri dish for 6 mins, rinsed with 2-propanol, and blown dry in a stream of nitrogen. The dried slides were placed on a hot plate (825-HP, VWR, West Chester, Pa.) for 2 hrs at 95° C. to perform the hard bake. After cooling, the slides were inspected under a microscope to confirm the 1002F base fabrication was achieved before fabrication of the cup edge.

Prior to the second spin cycle, the arrays were treated for 15 mins in a plasma cleaner (Harriclyck, Ithaca, N.Y.) to generate a hydrophilic surface, thus preventing air bubbles from being trapped between the base structures during the second photoresist spin cycle. After surface treatment, SU8-100 was spun on the slides in a similar manner as the 1002F except that a spin speed of 1800 rpm was used in the second spin step. The spun slides were then baked in the convection oven at 95° C. for 25 mins to remove the organic solvent in SU8. After cooling, the slides were aligned to the chrome mask for the cup edge and UV exposed using a mask aligner (MA6, SUSS Microtec, Germany). The arrays were then subjected to a PEB of 10 mins at 95° C., and then allowed to cool to room temperature over a 45 min period. The developing process after exposure was a two step process. The initial step was carried out in a bath of SU8 developer for 5 mins followed by a 7 min SU8 developer spray using a squeeze bottle. The slides were then rinsed with isopropyl alcohol followed by deionized water and dried on a hotplate at 95° C. for 1 hr. A reservoir was created for each array using molded PDMS which was then glued to the glass substrate with a thin layer of PDMS (FIG. 6 A).

Electron microscopy of microcups. The fabricated microcups were observed using an environmental scanning electron microscope (ESEM) (FEI Quanta 200, FEI Company, Hillsboro, Oreg., USA). The ESEM was performed in low vacuum (0.75 Torr) mode and a backscattered electron detector (BSED) was chosen to take images of the microcup. With the utilization of the ESEM, the microcup array could be directly observed without the surface metallic sputtering step, which is necessary when using, normal scanning electron microscope (SEM).

Laser-based microcup release. Release of microfabricated elements similar to the microcups using a laser-based method has been previously described (G. T. Salazar, et al., J. Biomed. Opt., 2008, 13, 034007; Y. Wang et al., Anal. Chem., 2007, 79, 2359-2366). Briefly, a laser pulse (5 ns, 532 nm) from a Q-switch Nd:YAG laser (Minilite I, Continuum Electro-Optics Inc., Santa Clara, Calif.) was focused by the microscope objective at the interface of the microcup base and glass substrate. Each focused pulse leads to formation of a plasma and cavitation bubble. The expansion of the cavitation bubble between the base of the microcup and glass substrate mechanically dislodged the microcup (P. A. Quinto-Su et al., Anal. Chem., 2008, 80, 4675-4679). To minimize the laser energies used to release an individual microcup, a series of pulses directed at different areas of the microcup base was used (energy per pulse=3 μJ, avg. pulse number=10) as described previously.

Cell culture. Adherent cells (HeLa, a human ovarian carcinoma cell line) and nonadherent cells (Ba/F3, a murine B cell lymphoma cell line) were used in the experiments. Two types of HeLa cells were utilized: non-fluorescent wild-type HeLa cells and a HeLa cell line possessing fluorescent nuclei due to stable transfection with a fusion protein of histone-H1 and green fluorescent protein (GFP). Medium for cell culture was prepared by adding. FBS (10%), L-glutamine (584 mg/L), penicillin (100 units/mL) and streptomycin (100 μg/L) to 500 mL of DMEM (Hela cells) or RPMI (Ba/F3 cells). Before loading, cells, microcup arrays were sterilized by immersion in 75% ethanol for 5 mins. The ethanol was removed by aspiration and the arrays were rinsed seven times with phosphate buffered saline (PBS: 135 mM NaCl, 3.2 mM NaHPO4, 0.5 mM KH2PO4 and 1.3 mM KCl; pH=7.4). The microcup surface was coated with fibronectin to enhance attachment of HeLa cells. The coating protocol consisted of incubating the microcup array in 2 mL of 25 μg/mL fibronectin in water for 16 hrs at room temperature. The microcup chamber was then rinsed seven times with PBS. No fibronectin coating was used when Ba/F3 cells were plated. To plate cells on the array, cells were suspended in the appropriate media and were added to the array chamber and allowed to settle. Cells plated on the array were cultured in a humidified, 5% CO₂ atmosphere at 37° C.

Viability assay. HeLa cells were plated and cultured on the microcup array in the incubator for 72 hrs. The array was washed five times with buffer (135 mM NaCl, 5 mM KCl, 10 mM HEPES, 2 mM MgCl₂, and 2 mM CaCl₂, pH 7.4). The cells were loaded with the fluorescent viability dye Calcein Red-Orange per manufacturer's protocol (2 μM, Invitrogen, Carlsbad, Calif.). After washing in buffer, the array was imaged by transmitted light and fluorescence microscopy. Cells were scored as viable (fluorescent) or non-viable (non-fluorescent).

Cell collection after microcup release. Prior to laser release of microcups, the array was rinsed with fresh culture medium three times to remove non-adherent cells. After laser-based release, microcups were collected by simply pipeting the media overlying the array with a 10 mL serological pipet and transferring to a 35 mm Petri dish under sterile conditions. The collected cells were then maintained in freshly prepared conditioned media for expansion.

### RESULTS

Structure of microcups. Shown in FIG. 6 is the scheme for the two step fabrication of the microcup array. Unless otherwise specified, the cup base was 70-100 μm on edge with a height of 50 μm. The height of the wall forming the cup was 40 μm. The gaps between the individual microcups and the thickness of the cup wall were varied to optimize the capture efficiency as described below. Evaluation of the structure of fabricated microcups was performed by imaging with transmitted light microscopy and ESEM to confirm their dimensions and the correct alignment of the base and wall (FIG. 1 B&C).

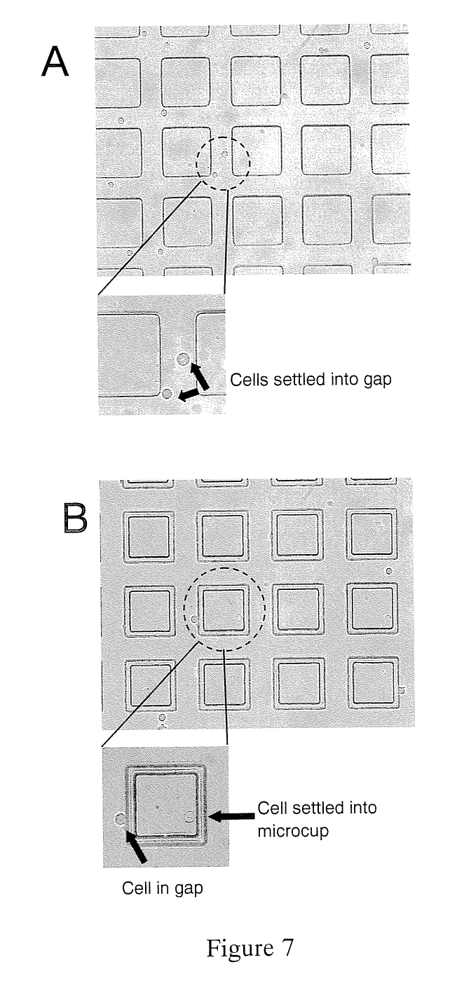

Capture of cells within the microcups. A series of experiments was performed to study the capture and confinement of cells plated on the microcup array. To study the capture of cells during plating, a suspension of Ba/F3 cells, a B lymphocyte cell line which grows in a nonadherent manner, was used (M. Warmuth et al., Curr. Opin. Oncol., 2007, 19, 55-60). The cells used in this study possessed a mean diameter of 15 μm±5 (n=120). To compare cell loading, suspensions of Ba/F3 cells (n=15,000, V=1 ml) were added to a microcup array (cup size 100 μm, wall thickness 10 μm, gap 50 μm, total number of microcups on the array=15,000) or a standard micropallet array (pallet size 100 μm micropallet, gap 50 μm, total number of pallets on the array=15,000). In these studies, no virtual wall was present and media filled the regions between elements on the array. After loading the cell suspension, the arrays were placed in an incubator for 20 mins to allow settling of the cells followed by microscopic observation under transmitted light. When using the standard pallet array with media rather than air walls present in the gaps, nearly all cells (98%) settled between pallets (FIG. 7A) as has been shown previously (J. R. Kovac and J. Voldman, Anal. Chem. 2007, 79, 9321-9330). In contrast, when using the microcup array, cells settled both inside the microcups and in the gap (FIG. 7B). Under the conditions used, the percentage of cells in the gap region was reduced to 40±2% (n=100). These results suggested that it was feasible to use the microcup design to replace the function of air (or PEG) in confining cells to the pallets.

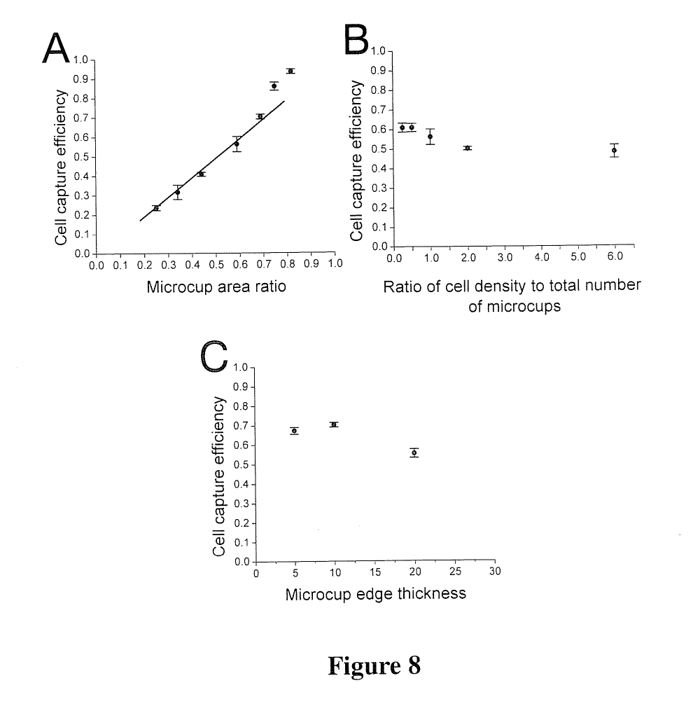

Capture efficiency of microcup arrays. In using the microcup array for culture and isolation of cells, cells must be captured within the microcups. Optimization of the capture efficiency was desired in order to minimize sample size requirements for a given array size. A series of experiments were undertaken to assess and optimize the capture efficiency of the array. To test if the total number of cells loaded onto the array affected the capture efficiency, Ba/F3 cell suspensions of varying cell densities were loaded onto microcup arrays (cup size 100 μm, wall thickness 10 μm, gap 30 μm). The cell densities were chosen relative to the total number of microcups making up the array. Six different cell densities were chosen as ¼, ½, 1, 2 and 6 times of the number of microcups (15,000) covering a range of sample sizes from 3,750 to 60,000 cells. Over this range, the capture efficiency remained fairly consistent at 50-60% (FIG. 8A).

It was reasoned that reducing the gap between microcups to maximize the coverage area of microcups over the array surface would enhance the capture efficiency. In these experiments the microcup area ratio was defined as the area of the array covered by the microcups vs. the total area of the array. Multiple arrays were designed having a constant number of microcups (15,000) of consistent size (cup size 100 μm, wall thickness 10 μm), but with gaps that varied between the individual arrays. The gaps tested were 100 μm, 70 μm, 50 μm, 30 μm, 20 μm, 15 μm, and 10 μm which corresponded to microcup area ratios of 25%, 34%, 44%, 59%, 69%, 76%, and 82%, respectively. Ba/F3 cell suspensions with constant cell numbers (15,000) were loaded on the arrays and allowed to settle. The capture efficiency defined as the number of cells settling within the microcups vs. the total number of cells present in the field of evaluation was then determined for each array (FIG. 8B). The capture efficiency linearly increased as the microcup area ratio increased; however, when the gap between microcups was similar to, or less than, the cell diameter (≦15 μm), the capture efficiency:microcup area ratio no longer followed a linear relationship. The capture efficiency increased to 86±2% (n=100) and 94±1% (n=100) for gaps of 15 μm and 10 μm, respectively. The capture efficiency for 10 μm gaps did not reach 100% as some cells settled within the region bounded by the corners of four adjacent microcups or on the boundary walls of adjacent microcups (see below). To test if the thickness of the microcup wall affected the cell capture efficiency, arrays with microcups of 100 μm diameters and gaps of 20 μm were fabricated with wall thicknesses of 5 μm, 10 μm and 20 μm. Ba/F3 cell suspensions were plated as above on the arrays and the capture efficiencies were determined (FIG. 3C). While no major differences were noted as a function of wall thickness, a few cells were observed to settle on the top of the 20 μm wide microcup edge without settling into the microcups or within the gaps (data not shown) suggesting that the microcup walls should be less than 20 μm in thickness to prevent this occurrence. These experiments indicated that gap size is the critical factor in designing the microcup arrays to maximize cell capture efficiency; however, gaps of less than 15 μm produce challenges in alignment and fabrication that place practical limits on the achievable gap dimensions.

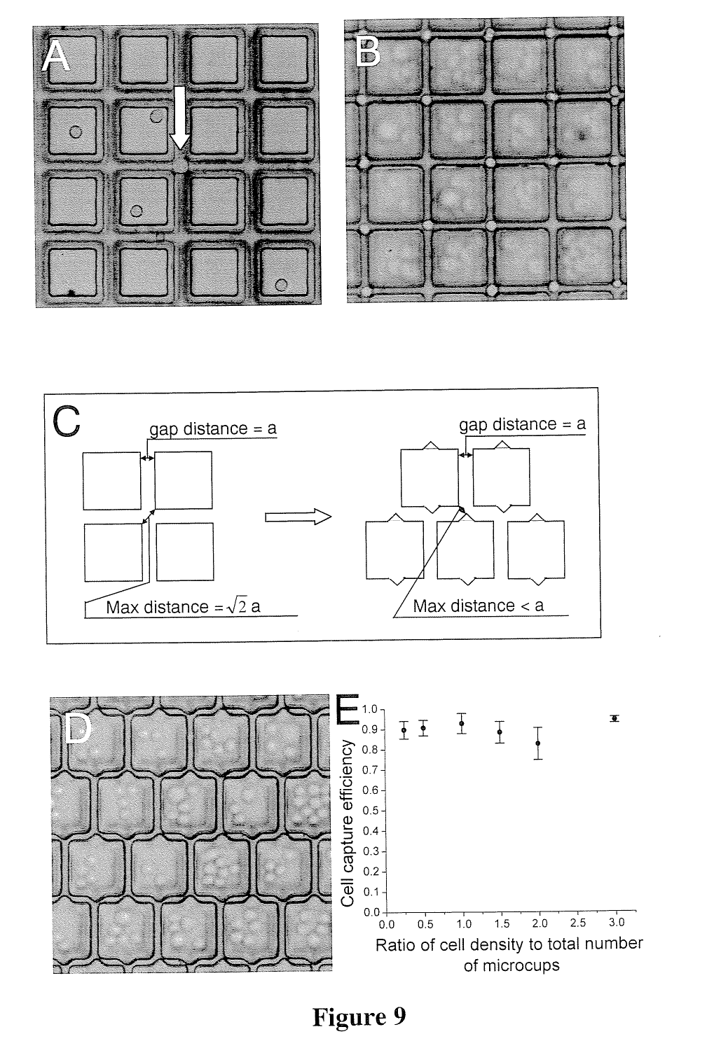

Improved microcup design. In hopes of improving the capture efficiency of the microcup array, the shape of the microcup was redesigned. For these experiments, the microcup size was reduced to 70 μm while maintaining a gap of 15 μm. These dimensions resulted in a reduced microcup area ratio of 68% compared with the 100 μm/15 μm microcup/gap array above (microcup area ratio=76%). To determine an appropriate redesign of the microcup shape, the pattern by which cells settled onto an array composed of standard square microcup shape (cup size=70 μm, wall thickness=10 μm, gap=15 μm) was studied. The capture efficiency of this array for a suspension of Ba/F3 cells (15,000) was 77%±2% (n=100). In evaluating the location of settled cells, it was found that the region bounded by the four corners of adjacent microcups was a sink for trapped cells with 45±10% (n=100) of those cells not captured by the microcups present in these regions. In addition, some cells whose diameter was larger than 15 μm were seen to be present in this region (FIG. 9A). At higher cell suspension density (8× the total number of microcups), up to 83±8% (n=100) of those cells settling outside the microcups were found in these regions, and frequently more than one cell could be found in a single region (FIG. 9B). The cause of this problem was due to the fact that the central distance across this region was ˜1.4 times greater than the gap size (as illustrated in FIG. 4C). If the gap size was 15 μm, the maximum distance across the center of this region would be ˜20 μm. If the resolution of mask printing and microfabrication was considered, the corner of the microcup could not be as pointed as designed on the mask file, and so it was likely that this distance was greater than 20 μm. Thus, these regions were more likely to trap multiple or larger cells than the gap regions along the sides of the microcups. While further reduction of the gap could help to reduce the dimension of this region, the challenge of microfabricating such closely spaced elements becomes a limiting factor. It was reasoned that reduction of this problematic area could be accomplished through redesign of the shape and registration of the microcups without increasing the complexity of microfabrication as illustrated in FIG. 9C. In this design, the dimension of the central aspect of this region would be less than the gap. An array of microcups of equivalent dimensions and area ratio (cup size 74 μm, gap 15 μm, area ratio 68%) was then successfully fabricated using the identical microfabrication process as for the square microcups. Cell capture efficiency was then assessed using Ba/F3 cells as above. With the newly designed arrays, no cells were observed in the regions bounded by three microcups, even at a cell density 8-fold greater than the total number of microcups (FIG. 9D). Furthermore, despite the decrease in microcup area ratio, the cell capture efficiency in the new design array was increased from 77% to 90%±4% (n=100, FIG. 9).

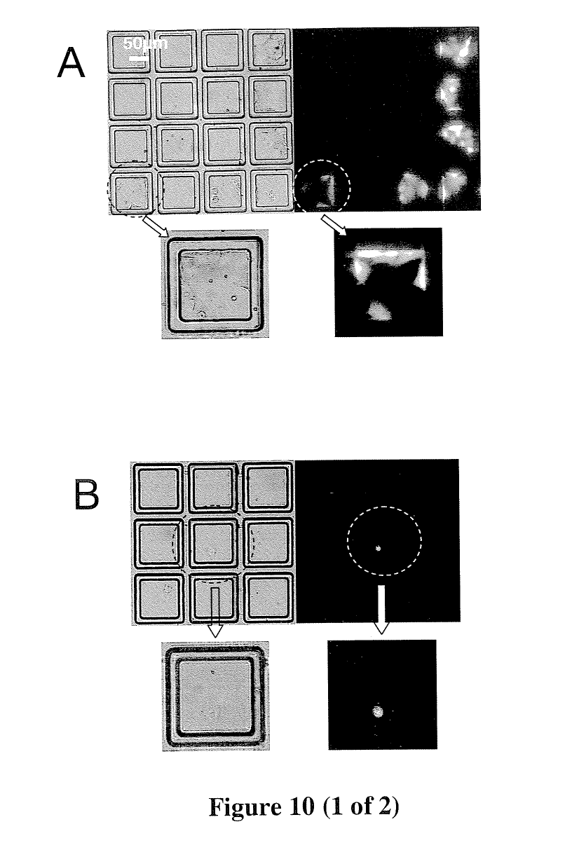

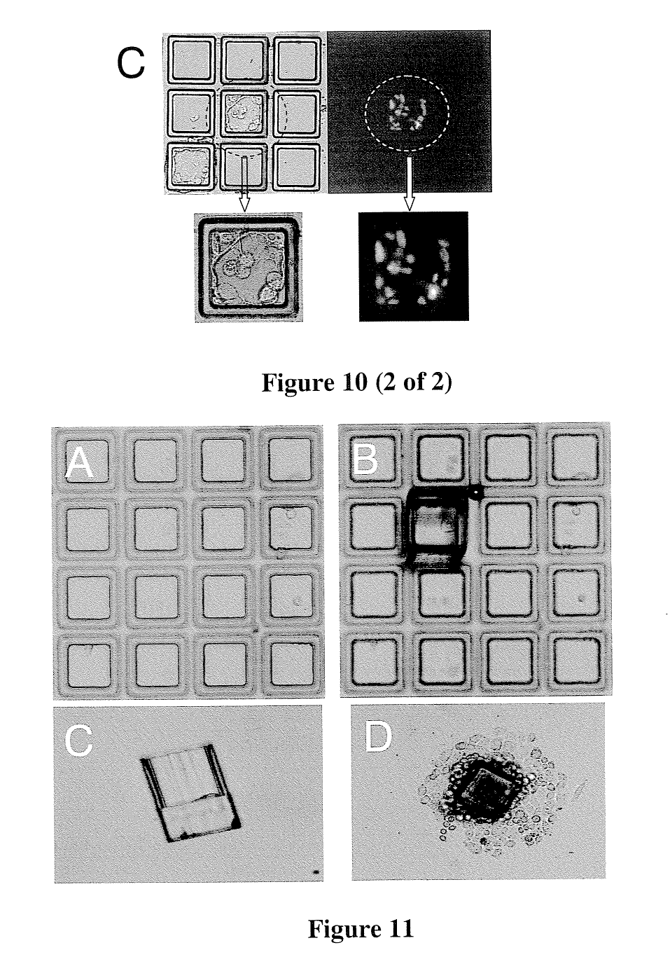

Culture of cells on the microcup arrays. Although cells cultured in microcups appeared to be healthy based on their morphology, cell viability was formally tested by viability dye and by assessing cell growth on the arrays. HeLa cells in suspension (10³) were plated on a microcup array (cup size 100 μm, wall thickness 10 μm, gap 50 μm, n_{microcup}=1,200) and placed under standard tissue culture conditions for 72 hrs. While still adherent within the microcups, the cells were labeled with the viability dye Calcein Red-Orange and observed by transmitted light and fluorescence microscopy. The result (FIG. 10) demonstrated 100% of HeLa cells cultured on the microcup array remained viable.

Expansion of single cells on the microcup provided both an indication of cell health and a measure of the ability of the microcups to sequester captured cells over time. For these experiments HeLa cells stably transfected with a GFP/histone-H1 fusion protein were mixed 1:10 with wild-type HeLa cells and were plated (10³ total cells) on an identical array as that described in the previous paragraph. The number of cells plated was arrived at empirically to provide a majority of individual microcups possessing either 1 or 0 cells. The GFP/histone-H1 HeLa cells possessed fluorescent nuclei while the wild-type HeLa cells were non-fluorescent. Cell expansion and sequestration was tested by following single HeLa cells having, a fluorescent nucleus (n=5) residing in microcups that were surrounded either by empty microcups or microcups containing only wild-type HeLa cells. To ease identification and tracking of the individual microcups, row numbers and column numbers were fabricated on the edges of the array used in this test. The array was imaged 1 hr after plating, by bright field and fluorescence microscopy to identify target cells. The array was then placed under standard tissue culture conditions and followed at 24 hr intervals to track growth of the fluorescent cells within the microcups. Each target cell was seen to grow into a clonal colony as assessed by the presence of fluorescent nuclei in all daughter cells within the microcup (FIG. 10 B&C). Importantly, the daughter cells remained sequestered for the duration of the experiment with an average of 16±5 cells present in the microcups at the 96 hr time point. These results clearly show that cells remain viable and sequestered during culture on the microcup arrays. Sequestration of a rapidly growing cell line such as HeLa for at least 4 days compares very favorably with literature in which cell arrays based on chemical surface modifications retained this cell type within the patterned cell islands for less than 72 hrs (D. I. Rozkiewicz, Y. Kraan, M. W. Werten, F. A. de Wolf, V. Subramaniam, B. J. Ravoo and D. N. Reinhoudt, Chemistry, 2006, 12, 6290-6297).

Isolation of single cells with the microcups. To determine if single cells could be isolated in a viable manner and clonally expanded, wild-type HeLa cells were plated on a microcup array. Microcups with single cells were identified and released (FIG. 6 A&B) using a low energy laser pulse protocol. Following release, the detached microcups were collected. As shown in FIG. 11C&D, the cells (n=7) remained attached and could easily be transferred using a standard pipette into a 35 mm diameter Petri dish for further expansion. After 13 days in culture, 100% of single cells collected in this manner grew into clonal colonies. The 3-dimensional structure should provide enhanced protection to cells during the collection process by virtue of the walls surrounding and shielding the cells. This statement is supported by results from our previous attempts to transfer cells by pipet after release from standard micropallet arrays. Those efforts were routinely unsuccessful due to loss of cells from the pallets during transfer. These data using cells collected in microcups clearly demonstrate the practicality of viable cell isolation and single-cell cloning using the microcup array.

The foregoing is illustrative of the present invention, and is not to be construed as limiting thereof. The invention is defined by the following claims, with equivalents of the claims to be included therein.

1. In an apparatus for collecting or culturing cells or cell colonies, said apparatus comprising a common substrate and a plurality of cell carriers releasably connected to said common substrate, with said carriers arranged in the form of an array, the improvement comprising:
employing microcups as said cell carriers, each of said microcups comprising a base portion and a top portion, said top portion having a cavity configured to contain cells formed therein;
and wherein said apparatus is essentially free of walls or barriers formed on said common substrate between said cell carriers.

2. The apparatus of claim 1, wherein said microcups are transparent.

3. The apparatus of claim 1, wherein said microcups in said array are separated by gaps, and wherein said gaps have an average width of from 2 to 200 micrometers;
and wherein said gaps have an average width of not more than 1000 micrometers.

4. The apparatus of claim 1, wherein said substrate is uncoated and underivatized.

5. The apparatus of claim 1, wherein said microcups have heights in the range of 2 micrometers up to 500 micrometers;
and wherein said microcups have maximum widths in the range of 5 micrometers up to 1000 micrometers.

6. The apparatus of claim 1, wherein said cavity has a depth of from 1 to 200 micrometers;
and wherein said cavity has a minimum width of at least 1 micrometer and a maximum width of less than 1000 micrometers.

7. The apparatus of claim 1, wherein said cavity is cylindrical, elliptical, triangular, rectangular, pentagonal, or hexagonal in shape.

8. The apparatus of claim 1, wherein said cavity comprises side wall portions, and wherein said side wall portions are parallel, inwardly tapered, or outwardly tapered.

9. The apparatus of claim 1, wherein said cavity comprises side wall portions, and said side wall portions have at least one cell-retaining member formed thereon.

10. In a method of collecting or culturing cells or cell colonies by (a) depositing a liquid media carrying said cells on an apparatus comprising a common substrate and a plurality of cell carriers releasably connected to said common substrate, with said carriers arranged in the form of an array, and then (b) permitting or allowing said cells to settle on or adhere to said cell carriers, the improvement comprising:
employing microcups as said cell carriers, each of said microcups comprising a base portion and a top portion, said top portion having a cavity configured to contain cells formed therein
and wherein said array is a barrier-free array wherein said apparatus is essentially free of walls or barriers formed on said common substrate between said cell carriers.

11. The method of claim 10, wherein said cells are deposited on said apparatus at an efficiency of capture of at least 50 percent.

12. The method of claim 10, wherein said cells are non-adherent cells.

13. The method of claim 10, wherein said cells are non-adherent cells selected from the group consisting of hybridomas, lymphocytes, stem cells, egg cells or oocytes, gram negative bacteria, and gram positive bacteria, yeast and fungi.

14. The method of claim 10, wherein said microcups are transparent.

15. The method of claim 10, wherein said microcups in said array are separated by gaps, and wherein said gaps have an average width of from 2 to 200 micrometers;
and wherein said gaps have an average width of not more than 5, 10, 100, 500, or 1000 micrometers.

16. The method of claim 10, wherein said array is in the form of an interdigitated array.

17. The method of claim 10, wherein said microcups have heights in the range of 2 micrometers up to 500 micrometers;
and wherein said microcups have maximum widths in the range of 5 micrometers up to 1000 micrometers.

18. The method of claim 10, wherein said cavity has a depth of from 1 to 200 micrometers;
and wherein said cavity has a minimum width of at least 1 micrometer and a maximum width of less than 1000 micrometers.

19. The method of claim 10, wherein said cavity is cylindrical, elliptical, triangular, rectangular, pentagonal, or hexagonal in shape.

20. The method of claim 10, wherein said cavity comprises side wall portions, and wherein said side wall portions are parallel, inwardly tapered, or outwardly tapered.

21. The method of claim 10, wherein said cavity comprises side wall portions, and said side wall portions have at least one cell-retaining member formed thereon.

22. The apparatus of claim 1, wherein said cavity comprises a side wall portion, and wherein said side wall comprises a porous wall configured to retain cells therein and permit nutrients to pass therethrough.

23. The method of claim 10, wherein said cavity comprises a side wall portion, and wherein said side wall comprises a porous wall configured to retain cells therein and permit nutrients to pass therethrough.
